(19) United States
(12) Patent Application Publication
(10) Pub. No.: US 2025/0318939 A1
(43) Pub. Date: Oct. 16, 2025

(54) DOUBLE-LEAF SPRING STENT

(71) Applicant: Asahi Intecc Co., Ltd., Seto-shi (JP)

(72) Inventor: Hirobumi Watanabe, Irvine, CA (US)

(21) Appl. No.: 19/174,480

(22) Filed: Apr. 9, 2025

Related U.S. Application Data
(60) Provisional application No. 63/632,193, filed on Apr. 10, 2024.

(51) Int. Cl. A61F 2/90; A61L 31/02

(52) U.S. Cl. CPC A61F 2/90; A61L 31/022; A61F 2210/0014; A61F 2230/0069; A61L 2400/16

(57) ABSTRACT

Embodiments of the present disclosure relate to an expandable stent and method for using same. An example expandable stent includes a spring structure configured to exert a collapsed radial force in a collapsed state; exert an expanded radial force in an expanded state, the collapsed radial force being greater than the expanded radial force; and exert a peak radial force during a transition of the spring structure between the collapsed state and the expanded state, wherein the peak radial force is greater than the collapsed radial force and greater than the expanded radial force. The example spring structure may include a plurality of double-leaf springs.

### CROSS REFERENCE TO RELATED APPLICATIONS

This application claims the benefit of and priority to U.S. Application No. 63/632,193, filed Apr. 10, 2024, entitled “DOUBLE-LEAF SPRING STENT,” the disclosure of which is incorporated herein by reference in its entirety.

### TECHNICAL FIELD

This application generally relates to expandable stents and methods for using the same.

### BACKGROUND

Shape-changing stents may be used to facilitate surgical operations within tortuous structures, such as arteries, veins, and/or the like. For example, mechanical thrombectomy for stroke treatment may utilize a stent-retriever and aspiration catheter. The stent-retriever and aspiration catheter may be deployed within a blood vessel to enable aspiration of hazardous material (e.g., thrombus) from a target site. Existing approaches to aspiration-based thrombectomy have increased the bore size of the aspiration catheter to improve success rates of thrombus removal. To increase the bore size of the device, many approaches implement a covered stent. For example, a covered self-expanding stent may be delivered to a target site and unsheathed to significantly increase the bore size of the aspiration device relative to the delivery catheter, after which the thrombus may be aspirated through the expanded stent into the aspiration device.

However, tradeoffs between device deliverability and bore size may inhibit the effective use of such an aspiration device in, for example, tortuous neurovascular environments. For example, an expandable stent with lower radial force may demonstrate greater deliverability through narrow, twisting spaces; however, a stent with lower radial force may be insufficient for withstanding the vacuum pressures generated by a hyper-bore-size aspiration device at the target site. Thus, existing approaches have yet to solve the challenge of resolving radial force requirements for enabling device deliverability to a tortuous target site while providing sufficient strength to, for example, resist negative pressures generated at the target site.

### BRIEF SUMMARY

Embodiments of the present disclosure relate to expandable stents, stent kits, and methods for using the same. An example stent of the present disclosure may include a spring structure, the spring structure configured to exert a collapsed radial force in a collapsed state; exert an expanded radial force in an expanded state; and exert a peak radial force during a transition of the spring structure between the collapsed state and the expanded state. The collapsed radial force is greater than the expanded radial force. The peak radial force is greater than the collapsed radial force and greater than the expanded radial force.

In some embodiments, the spring structure comprise a plurality of double-leaf springs. In some embodiments, a respective double-leaf spring comprises a first curved arm; a second curved arm; a connection between respective first ends of the first curved arm and the second curved arm; a first vertical arm connected to a second end of the first curved arm; a second vertical arm connected to a second end of the second curved arm; and a longitudinal member connected to opposing ends of the first vertical arm and the second vertical arm. In some embodiments, on a first side, the respective double-leaf spring is connected to an adjacent double-leaf spring via the longitudinal member. In some embodiments, the longitudinal member of the respective double-leaf spring is connected, at the opposing ends, to a first vertical arm and a second vertical arm of the adjacent double-leaf spring.

In some embodiments, on a second side opposite the first side, the respective double-leaf spring is connected to a second adjacent double-leaf spring via the connection between the respective first ends of the first curved arm and the second curved arm. In some embodiments, the first curved arm, second curved arm, first vertical arm, second vertical arm, and longitudinal member are integrally formed. In some embodiments, in the expanded state, the first vertical arm and the second vertical arm are orthogonal to the longitudinal member. In some embodiments, the plurality of double-leaf springs are integrally formed. In some embodiments, the spring structure comprises nitinol.

Another example stent of the present disclosure may include a plurality of rows of double-leaf springs in an annular arrangement; a respective row of double-leaf springs comprising an upper segment of double-leaf springs and a lower segment of double-leaf springs, wherein a respective double-leaf spring of the upper segment is connected to a corresponding double-leaf spring of the lower segment; and the double-leaf springs of a respective segment are connected to a first side of a longitudinal member that defines a length of the respective row; and the longitudinal member is connected to respective double-leaf springs of a respective upper or lower segment of another row of double-leaf springs.

In some embodiments, a respective double-leaf spring comprises a first curved arm; a second curved arm; a connection between respective first ends of the first curved arm and the second curved arm, respective second ends of the first curved arm and the second curved arm defining a span of the double-leaf spring; a first vertical arm connected to the second end of the first curved arm; a second vertical arm connected to the second end of the second curved arm; and the respective longitudinal member, wherein the first vertical arm and the second vertical arm are connected to the longitudinal members at opposing ends of the span.

In some embodiments, the annular arrangement is configured to transition the stent between a collapsed state and an expanded state. In some embodiments, in the collapsed state, an angle between a respective vertical arm and the longitudinal member is obtuse. In some embodiments, in the expanded state, the angle between the respective vertical arm and the longitudinal member is orthogonal.

Another example stent of the present disclosure may include a plurality of annular sections, a respective annular section comprising a plurality of double-leaf springs in an annular arrangement; a respective double-leaf spring in a first annular section comprising a first curved arm; a second curved arm; a connection between respective first ends of the first curved arm and the second curved arm, respective second ends of the first curved arm and the second curved arm defining a span of the double-leaf spring; a first vertical arm connected to the second end of the first curved arm; a second vertical arm connected to the second end of the second curved arm; and the respective longitudinal member, wherein the first vertical arm and the second vertical arm are connected to the longitudinal members at opposing ends of the span; and the respective longitudinal members of the double-leaf springs in the first annular section extending along a remaining subset of the plurality of the annular sections such that corresponding double-leaf springs in the remaining subset are partially comprised of the longitudinal members.

In some embodiments, a first end of the stent comprises a first annular arrangement of flat surfaces spaced apart from one another; and a respective flat surface of the first annular arrangement is defined by the respective first vertical arm of the plurality of double-leaf springs in a first annular section of the plurality of annular sections. In some embodiments, a second end of the stent comprises a second annular arrangement of flat surfaces spaced apart from one another; and a respective flat surface of the second annular arrangement is defined by the respective second vertical arm of the plurality of double-leaf springs in a second annular section of the plurality of annular sections.

In some embodiments, the stent is radially symmetrical about a longitudinal axis extending centrally through the plurality of annular sections. In some embodiments, the stent comprises a non-linear radial force profile. In some embodiments, the stent comprises at least one of cupro nickel aluminum alloy, silico manganese alloy, or cupro zinc aluminum alloy. In some embodiments, in a respective double-leaf spring: the first curved arm defines a first leaf spring; the second curved arm defines a second leaf spring; the first vertical arm defines a first vertical spring; and the second vertical arm defines a second vertical spring.

An example stent kit may comprise one or more stents as described herein and shown in the accompanying figures. In some embodiments, the kit further includes a respective sheath configured to cover a stent to maintain the stent in a collapsed state. In some embodiments, the kit includes at least a first stent and a second stent, where the first stent comprises a first diameter in the expanded state, the second stent comprises a second diameter in the expanded state, and the second diameter exceeds the first diameter. In some embodiments, a respective double-leaf spring of the first stent comprises a first thickness, a respective double-leaf spring of the second stent comprises a second thickness, the second thickness is less than the first thickness. In some embodiments, the kit further includes one or more aspiration devices comprising the respective sheath and stent.

An example method of use for a stent (or kit) of the present disclosure may include aspirating a tubular structure within a subject. The example method may include navigating a guidewire through the subject into the tubular structure; deploying a respective sheath and at least one stent into a target site of the tubular structure via the guidewire, the at least one stent being contained within the sheath in the collapsed state; retracting the sheath to cause transition of the at least one stent from the collapsed state to the expanded state, wherein the transition of the at least one stent to the expanded state expands an internal bore of the at least one aspiration device; and aspirating material from the target site via negative pressurization of the expanded internal bore. The material may include a thrombus, foreign object, and/or the like.

In some embodiments, the method further includes retracting the at least one stent into an interior of the sheath to transition the at least one state to the collapsed state, wherein the transition of the at least one stent to the collapsed state causes a reduction of the internal bore of the aspiration device; and removing the aspiration device, the sheath, and the at least one stent from the subject via the guidewire.

### BRIEF DESCRIPTION OF THE FIGURES

Having thus described the embodiments of the disclosure in general terms, reference now will be made to the accompanying drawings, which are not necessarily drawn to scale, and wherein:

FIG. 1A shows a right perspective view of an example stent in an expanded state in accordance with some embodiments of the present disclosure;

FIG. 1B shows a right perspective view of an example stent in a collapsed state in accordance with some embodiments of the present disclosure;

FIG. 2A shows a left perspective view of an example stent in an expanded state in accordance with some embodiments of the present disclosure;

FIG. 2B shows a left perspective view of an example stent in a collapsed state in accordance with some embodiments of the present disclosure;

FIG. 3A shows a left-side view of an example stent in an expanded state in accordance with some embodiments of the present disclosure;

FIG. 3B shows a left-side view of an example stent in a collapsed state in accordance with some embodiments of the present disclosure;

FIG. 4A shows a right-side view of an example stent in an expanded state in accordance with some embodiments of the present disclosure;

FIG. 4B shows a right-side view of an example stent in a collapsed state in accordance with some embodiments of the present disclosure;

FIG. 5A shows a top view of an example stent in an expanded state in accordance with some embodiments of the present disclosure;

FIG. 5B shows a top view of an example stent in a collapsed state in accordance with some embodiments of the present disclosure;

FIG. 6A shows a bottom view of an example stent in an expanded state in accordance with some embodiments of the present disclosure;

FIG. 6B shows a bottom view of an example stent in a collapsed state in accordance with some embodiments of the present disclosure;

FIG. 7A shows a front view of an example stent in an expanded state in accordance with some embodiments of the present disclosure;

FIG. 7B shows a front view of an example stent in a collapsed state in accordance with some embodiments of the present disclosure;

FIG. 8A shows a back view of an example stent in an expanded state in accordance with some embodiments of the present disclosure;

FIG. 8B shows a back view of an example stent in a collapsed state in accordance with some embodiments of the present disclosure;

FIG. 9 shows an example double-leaf spring in accordance with some embodiments of the present disclosure;

FIG. 10 shows a transition sequence of an example double-leaf spring between expanded and collapsed states in accordance with some embodiments of the present disclosure;

FIG. 11 illustrates a chart of example radial force-diameter relationships of a conventional stent and a double-leaf spring stent in accordance with some embodiments of the present disclosure;

FIG. 12 shows example deformations of a conventional leaf spring stent and a double-leaf spring stent in accordance with some embodiments of the present disclosure;

FIG. 13 shows a right perspective view of an example stent in an expanded state in accordance with some embodiments of the present disclosure;

FIG. 14 shows a left perspective view of an example stent in an expanded state in accordance with some embodiments of the present disclosure;

FIG. 15 shows a left-side view of an example stent in an expanded state in accordance with some embodiments of the present disclosure;

FIG. 16 shows a right-side view of an example stent in an expanded state in accordance with some embodiments of the present disclosure;

FIGS. 17A-B show, respectively, a top view and a bottom view of an example stent in an expanded state in accordance with some embodiments of the present disclosure;

FIGS. 18A-B show, respectively a front view and a back view of an example stent in an expanded state in accordance with some embodiments of the present disclosure;

FIG. 19 shows a right perspective view of an example stent in an expanded state in accordance with some embodiments of the present disclosure;

FIG. 20 shows a left perspective view of an example stent in an expanded state in accordance with some embodiments of the present disclosure;

FIGS. 21A-B show, respectively, a left-side view and a right-side view of an example stent in an expanded state in accordance with some embodiments of the present disclosure;

FIGS. 22A-B show, respectively, a top view and a bottom view of an example stent in an expanded state in accordance with some embodiments of the present disclosure;

FIGS. 23A-B show, respectively a front view and a back view of an example stent in an expanded state in accordance with some embodiments of the present disclosure; and

FIG. 24 illustrates a flowchart of an example aspiration process in accordance with some embodiments of the present disclosure.

### DETAILED DESCRIPTION

Some embodiments of the present invention will now be described more fully hereinafter with reference to the accompanying drawings, in which some, but not all, embodiments of the invention are shown. Like reference numerals refer to like elements throughout. Indeed, various embodiments of the invention may be embodied in many different forms and should not be construed as limited to the embodiments set forth herein. Rather, these embodiments are provided so that this disclosure will satisfy applicable legal requirements.

As used herein, the term “or” is used in both the alternative and conjunctive sense, unless otherwise indicated. The term “along,” and similarly utilized terms, means near or on, but not necessarily requiring directly on an edge or other referenced location. The terms “approximately,” “generally,” and “substantially” refer to within manufacturing and/or engineering design tolerances for the corresponding materials and/or elements unless otherwise indicated. Thus, use of any such aforementioned terms, or similarly interchangeable terms, should not be taken to limit the spirit and scope of embodiments of the present invention.

As used herein, reference is made to a double-leaf spring stent for use in conjunction with an aspiration device. The present disclosure, however, contemplates that the double-leaf spring stent of the present disclosure may be equally applicable to other applications in which reduced radial force in the expanded stent state is advantageous. For example, the double-leaf spring stent may be used in other clot removal procedures, stent retrieval procedures, angioplasty procedures, ureteral procedures, aneurysm interventions, and/or the like.

### Overview

In general, various embodiments of the present disclosure provide improved designs for self-expanding stents. For example, the disclosure provides various embodiments for a self-expanding, double-leaf spring stent for use in aspiration thrombectomy procedures. It will be understood and appreciated that such context is provided by way of example and uses of the stent in additional contexts, such as with other medical procedures, are contemplated and within the scope of the invention.

As described above, existing stents for aspiration thrombectomies face challenges in providing sufficient radial force at the target site to hyper-expand a bore of the aspiration device and resist negative pressurization while preserving steerability of the aspiration device through tortuous structures within the body. For example, means for achieving a significant increase in aspiration bore size may reduce the ability of the device to be navigated through narrow veins and other tubular structures within the body. Use of a self-expanding covered stent with weaker radial forces may provide reductions in the device spatial profile, which increase device deliverability. For example, during device delivery, a smaller friction between the sheath and stent may be beneficial such that lower radial stent forces are favorable as compared to higher radial forces. However, higher radial stent forces may be favored at the delivery target site to enable the stent to withstand high negative pressures generated by the aspiration device.

Other approaches to stent expansions rely upon balloon catheters. For example, a stainless-steel stent may be expanded via inflation of a balloon catheter. However, such approaches plastically and permanently deform the stent and, as a result, removal of the stent may require additional mechanisms for recompressing the stent, further increasing the complication and bulk of the aspiration device. Thus, a self-expanding stent may be preferable for use in aspiration thrombectomies; however, challenges exist in balancing radial force of the stent such the sufficiently low friction may be achieved while delivering the aspiration device while preserving ability of the stent to hyper-expand the aspiration bore and withstand negative pressures used in the procedure.

To solve these issues and others, example implementations of embodiments of the present application may provide a double-leaf spring stent that provides for reduced radial forces when the stent is configured to a collapsed state during the device delivery phase and maintains sufficiently strong radial forces when the stent is configured to an expanded state during the aspiration phase. In various embodiments, the double-leaf spring stent includes a novel spring arrangement to achieve a decreased radial force in compression. For example, conventional self-expanding stents demonstrate a peak radial force when in the compressed state. In contrast, the present double-leaf spring stent demonstrates a peak transitional radial force during transition to the compressed state. For example, the compressed radial force exerted by the double-leaf spring stent when configured to the compressed state is less than the peak transitional radial force reached during the transition to the compressed state. In various embodiments, the novel spring arrangement includes a combination of two leaf-springs, referenced herein as a “curved arm” and a “vertical arm.” In some embodiments, the curved arm embodies a “weaker spring,” and the vertical arm embodies a “strong spring.” In various embodiments, stent compression initially deforms both “weaker leaf-spring” (curved arm) and “stronger leaf-spring” (vertical arm). Subsequently, the further compression relaxes only the “stronger leaf-spring,” resulting in the decreases in radial force.

In this manner, the double-leaf spring stent described hereafter improves deliverability of the aspiration device by providing reduced radial forces in compression while maintaining sufficiently strong radial forces in expansion. The reduced radial forces may result in lower friction between the aspiration device and the walls of tortuous structures through which the aspiration device is inserted. Further, the double-leaf spring structure retains sufficient internal space in compression such that membranes of the aspiration device are relaxed, further increasing steerability of the aspiration device through tortuous pathways. In various embodiments, the reduced radial forces of the compressed double-leaf spring stent enable delivery of the stent (and aspiration device containing the stent) via pushing means that are advantageous over pulling means, which are associated with additional mechanical complexity and bulk.

### Example Double-leaf Spring Stent

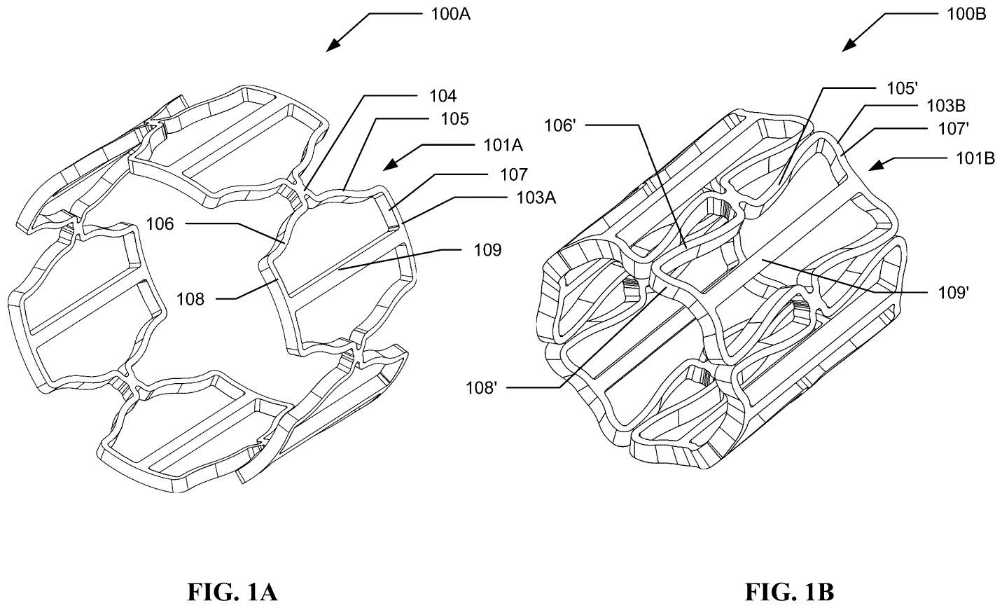

With reference to FIG. 1A, a right perspective view of an example stent 100A in an expanded state is illustrated. In some embodiments, the stent 100A includes a spring structure 101A configured to transition between an expanded state (e.g., as shown in FIG. 1A) and a collapsed state (e.g., as shown in the spring structure 101B of FIG. 1B). In some embodiments, the spring structure 101A is configured to exert an expanded radial force in the expanded state and exert a collapsed radial force in the collapsed state. In some embodiments, the collapsed radial force exceeds the expanded radial force. In various embodiments, the spring structure 101A is configured to exert a peak radial force during a transition between the collapsed state and the expanded state, where the peak radial force is greater than the collapsed radial force and greater than the expanded radial force.

In some embodiments, the spring structure 101A comprises nitinol, one or more nitinol-comprising alloys, and/or the like. Additionally, or alternatively, in some embodiments, the spring structure 101A comprises cupro nickel aluminum alloy, silico manganese alloy, cupro zinc aluminum alloy, and/or the like. In some embodiments, the spring structure 101A includes one or more radiopaque materials to increase visibility of the stent 100A under one or more radiological imaging modes.

In some embodiments, the spring structure 101A includes a plurality of double-leaf springs 103A. In some embodiments, the double-leaf springs are interconnected in an annular arrangement. In some embodiments, a respective double-leaf spring includes a first curved arm 105, second curved arm 106, first vertical arm 107, second vertical arm 108, and longitudinal member 109. In some embodiments, a first side of the double-leaf spring 103A includes the first curved arm 105 and the first vertical arm 107. In some embodiments, on a second side opposite the first side, the double-leaf spring 103A includes the second curved arm 106 and the second vertical arm 108. In various embodiments, the elements of the double-leaf spring 103A are integrally formed. For example, the first curved arm 105, second curved arm 106, first vertical arm 107, second vertical arm 108, and longitudinal member 109 may be integrally formed within one another. Further, in some embodiments, the plurality of double-leaf springs 103A embodying the spring structure 101A are integrally formed.

In various embodiments, the double-leaf spring 103A includes a connection 104 between the first curved arm 105 and the second curved arm 106. In some embodiments, the connection 104 includes a first side at which a first curved arm 105 and second curved arm 106 of a first double-leaf spring are connected. In some embodiments, the connection 104 includes a second side, opposite the first side, at which a first curved arm 105 and second curved arm 106 of a second double-leaf spring are connected.

In some embodiments, the first curved arm 105 is connected to a first end of the first vertical arm 107. In some embodiments, on a second end opposite the first end, the first vertical arm 107 is connected to a first end of the longitudinal member 109. In some embodiments, the second curved arm 106 is connected to a first end of the second vertical arm 108. In some embodiments, on a second end opposite the first end, the second vertical arm 108 is connected to a second end of the longitudinal member 109 (e.g., opposite the first end of the longitudinal member). In various embodiments, a respective curved arm defines a lateral leaf spring comprising variable curvature. The variable curvature may comprise multiple convex and concave portions (e.g., arches).

In various embodiments, at a first end (e.g., at the connection 104) a respective curved arm comprises a first concave portion. In some embodiments, adjacent the first concave portion, the curved arm comprises a first convex portion. In some embodiments, adjacent the first convex portion the curved arm comprises a second concave portion. In some embodiments, adjacent the second concave portion the curved arm comprises a second convex portion. The second convex portion may embody a connection between the curved arm and a respective vertical arm. In some embodiments, the arms are straight arms in the expanded state.

In some embodiments, in the expanded state, a respective vertical arm is oriented orthogonal to the longitudinal member 109. For example, a respective angle between the longitudinal member 109 and each of the first vertical arm 107 and the second vertical arm 108 may be orthogonal (e.g., approximately 90 degrees) while the spring structure 101A is configured to the expanded state. In some embodiments, a respective longitudinal member is connected to a first and second vertical arm of a first double-leaf spring on a first side. In some embodiments, on a second side opposite the first side, the longitudinal member 109 is further connected to a first and second vertical arm of a second double-leaf spring.

FIG. 1B shows a right perspective view of an example stent 100B in a collapsed state. In various embodiments, the stent 100B embodies the stent 100A transitioned from the expanded state shown in FIG. 1A to a compressed state. For example, the spring structure 101B may embody a contracted spring structure 101A. An outward radial force exerted by the spring structure 101B may exceed an outward radial force exerted by the spring structure 101A (e.g., said radial forces being referred to as a contracted radial force and an expanded radial force, respectively). A double-leaf spring 103B may embody a collapsed double-leaf spring 103A.

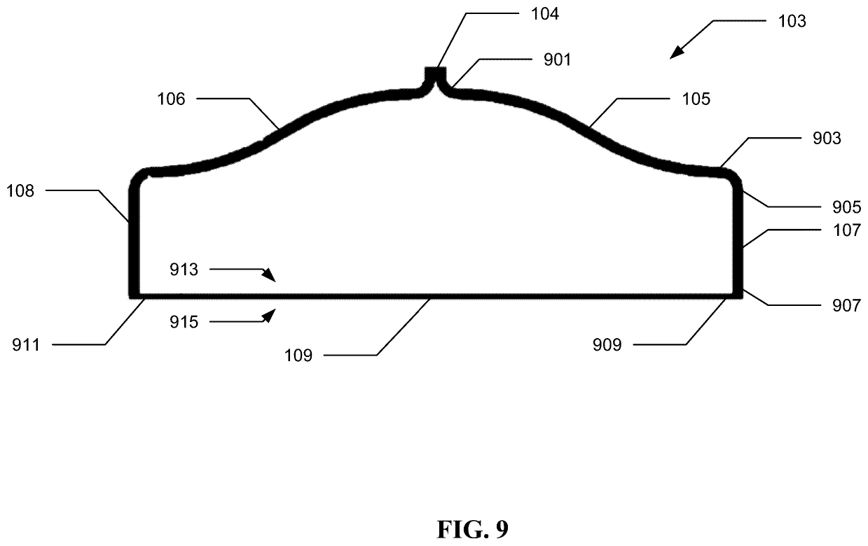

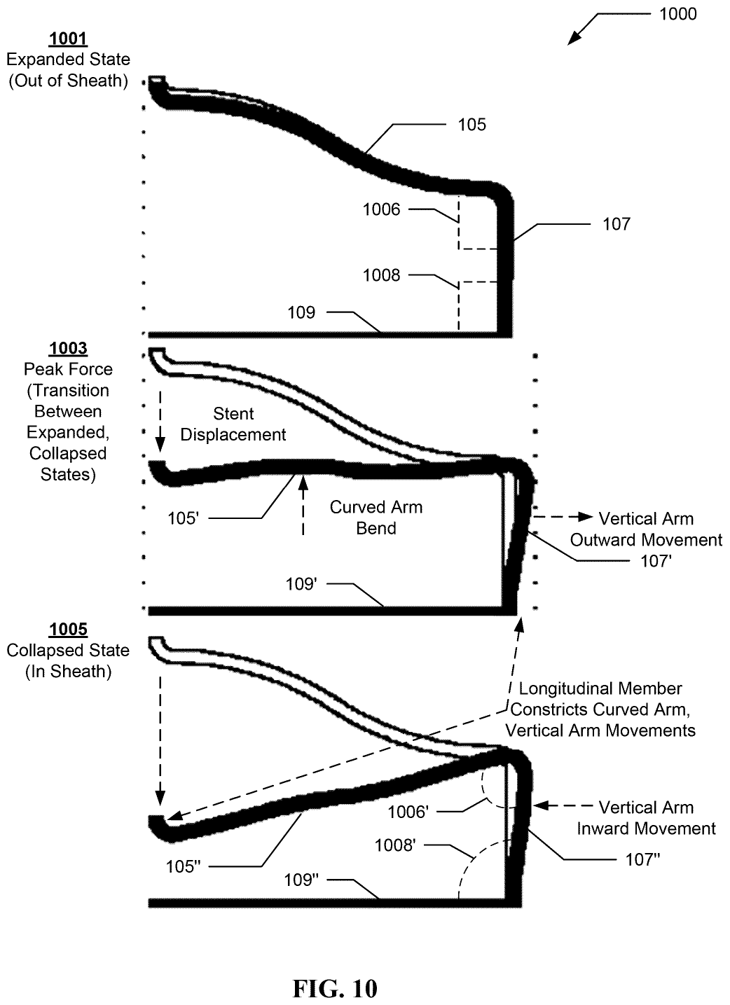

In some embodiments, in the collapsed state, a first curved arm 105′ and a second curved arm 106′ of the double-leaf spring 103B are deflected toward the longitudinal member 109′ relative to the orientations shown in FIG. 1A and further illustrated in FIG. 10. In some embodiments, in the collapsed state, one or more portions of a first vertical arm 107′ and second vertical arm 108′ are deflected outward relative to the orientation shown in FIG. 1A and FIG. 9. In various embodiments, the respective deflections of the first and second curved arms toward the longitudinal member and outward deflections of the first and second vertical arms are further depicted in the example transition sequence 1000 shown in FIG. 10 and described herein.

In some embodiments, during transitions between the collapsed and expanded states, the longitudinal member constricts the respective deflections of the first and second curved arms 105′, 106′ and the first and second vertical arms 107′, 108′. For example, the stent 100A may be transitioned to the configuration embodied as stent 100B via retraction of the stent into a sheath that applies an inward radial force causing collapse of the spring structure from the expanded state to the collapsed state. The inward radial force may cause deflection of the curved arms of the double-leaf springs comprising the spring structure, said deflection moving toward the respective longitudinal member of the corresponding double-leaf spring. In such contexts, the respective connections between the longitudinal member 109 and the first vertical arm 107′ and between the first vertical arm 107′ and the first curved arm 105′, the longitudinal member 109 may constrain deflection of the first curved arm 105′ to a maximum angle at which further compressive forces cause outward deflection of the first vertical arm 107′ (e.g., as opposed to causing further deflection of the first curved arm 105′ toward the longitudinal member 109′ beyond the maximum angle). In doing so, the stent 100A, B may demonstrate a non-linear trend in outward radial force exerted by the spring structure during transitions between expanded and collapsed states.

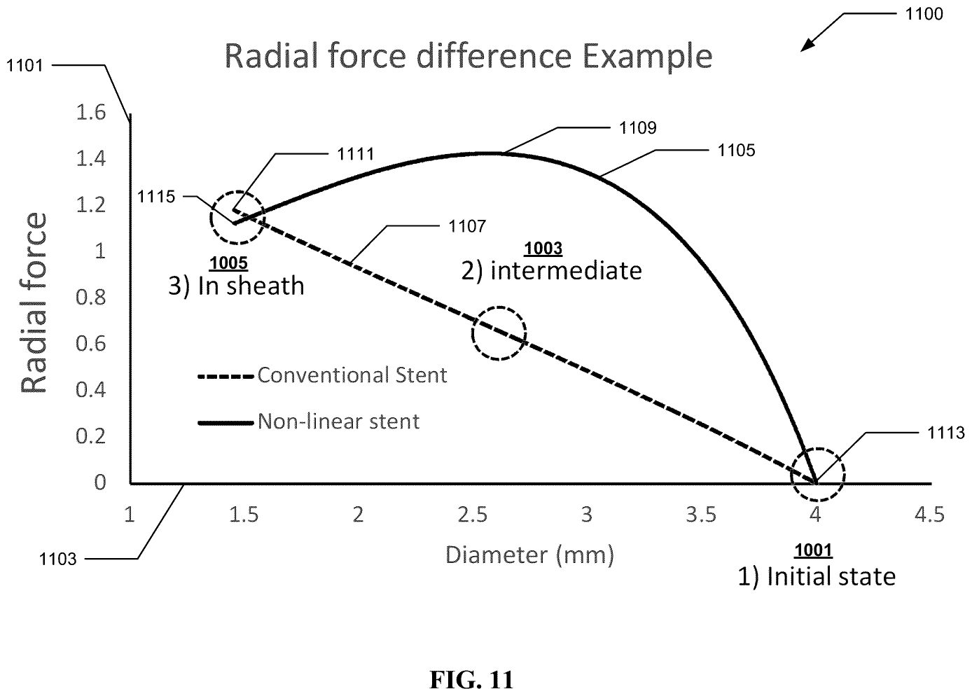

For example, as further depicted in the chart 1100 of FIG. 11, a typical self-expanding stent may demonstrate a linear relationship between outward radial force exerted and stent diameter. In contrast, the stent 100A, 100B (e.g., and other double-leaf spring stents of the present disclosure) may present a non-linear relationship between outward radial force exerted and stent diameter.

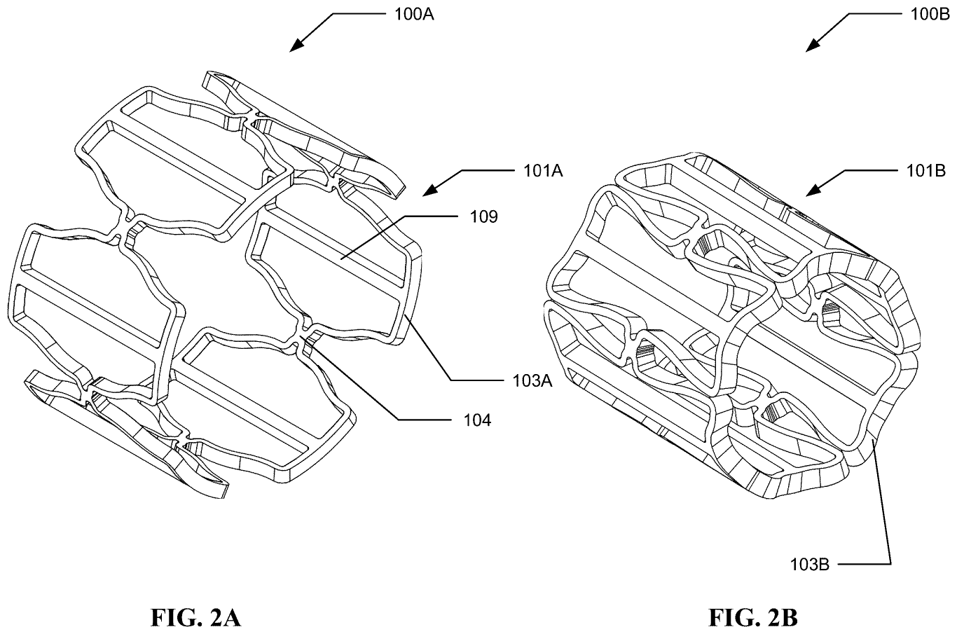

FIG. 2A shows a left perspective view of the example stent 100A in an expanded state. FIG. 2B shows a left perspective view of the example stent 100B in a collapsed state. As shown, the spring structure 101A includes a plurality of double-leaf springs 103A in an annular arrangement. In various embodiments, adjacent double-leaf springs are connected via a longitudinal member 109 or a connection 104. In some embodiments, the double-leaf springs embodying the spring structure are integrally formed. For example, on a first side, a first double-leaf spring 103A may be connected to a second double-leaf spring 103A via a shared longitudinal member 109. On a second side of the first double-leaf spring 103A, said first double-leaf spring 103A may be connected to a third double-leaf spring 103A via a connection 104. The second double-leaf spring 103A may be further connected to a fourth double-leaf spring 103A via another connection 104, and the third double-leaf spring 103A may be further connected to a fifth double-leaf spring 103A via another shared longitudinal member 109. The spring structure 101A may include an additional number of double-leaf springs connected in an annular sequence such that the spring structure comprises a substantially annular shape. For example, the spring structure 101A may include twelve double-leaf springs connected to one another in an annular sequence.

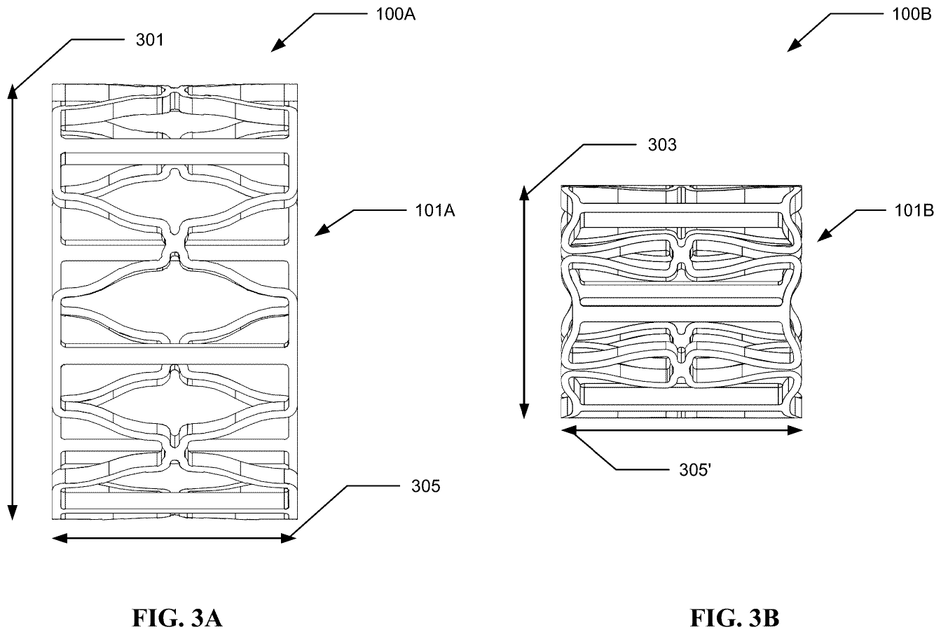

FIG. 3A shows a left-side view of the example stent 100A in an expanded state. FIG. 3B shows a left-side view of the example stent 100B in a collapsed state. As shown, the stent 100A, 100B includes a first diameter 301 and width in the expanded state and a second diameter 303 in the collapsed state. In some embodiments, the second diameter 303 is less than the first diameter 301. In various embodiments, the stent 100A, 100B includes a length 305, 305′ that remains constant in the collapsed and expanded states.

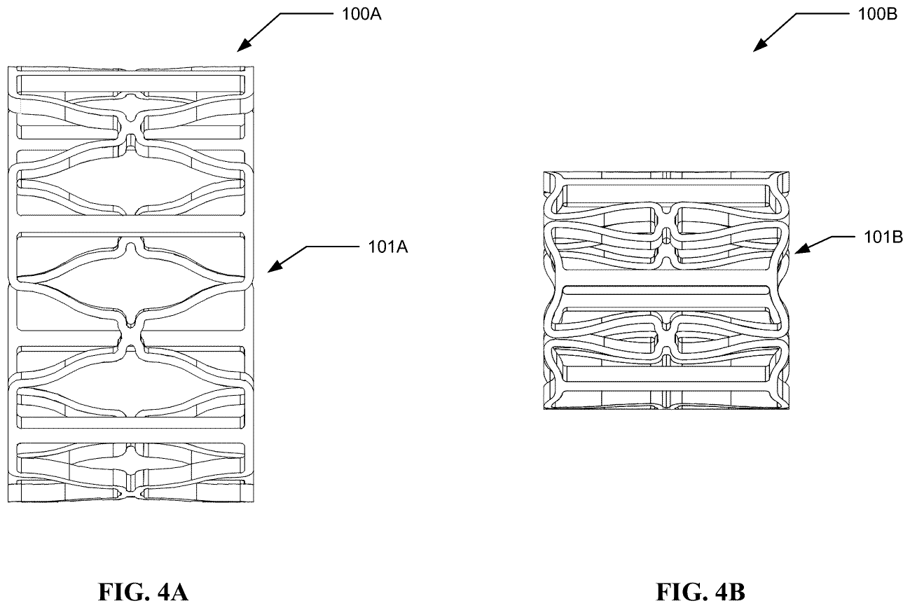

FIG. 4A shows a right-side view of the example stent in an expanded state. FIG. 4B shows a right-side view of the example stent 100B in a collapsed state.

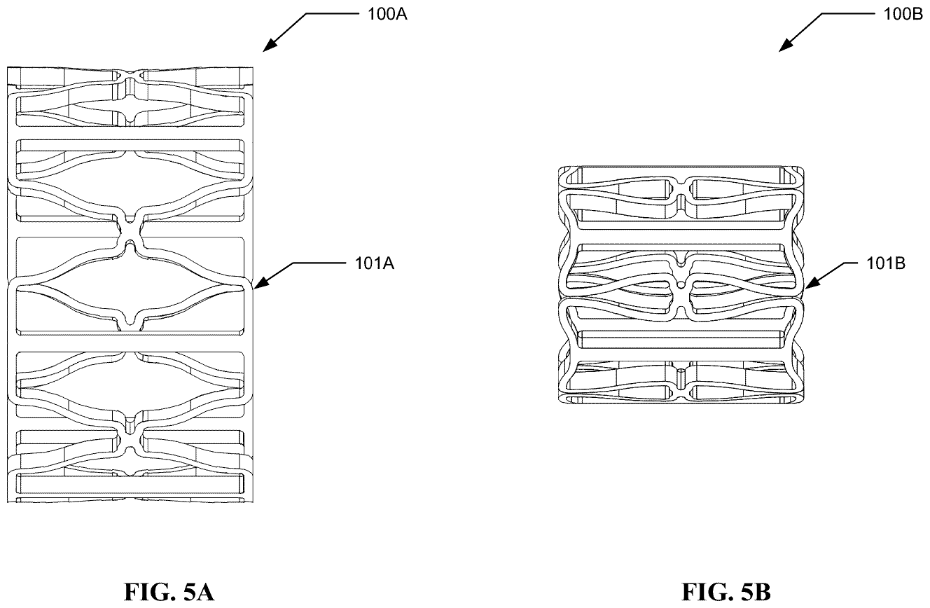

FIG. 5A shows a top view of the example stent 100A in an expanded state. FIG. 5B shows a top view of the example stent 100B in a collapsed state.

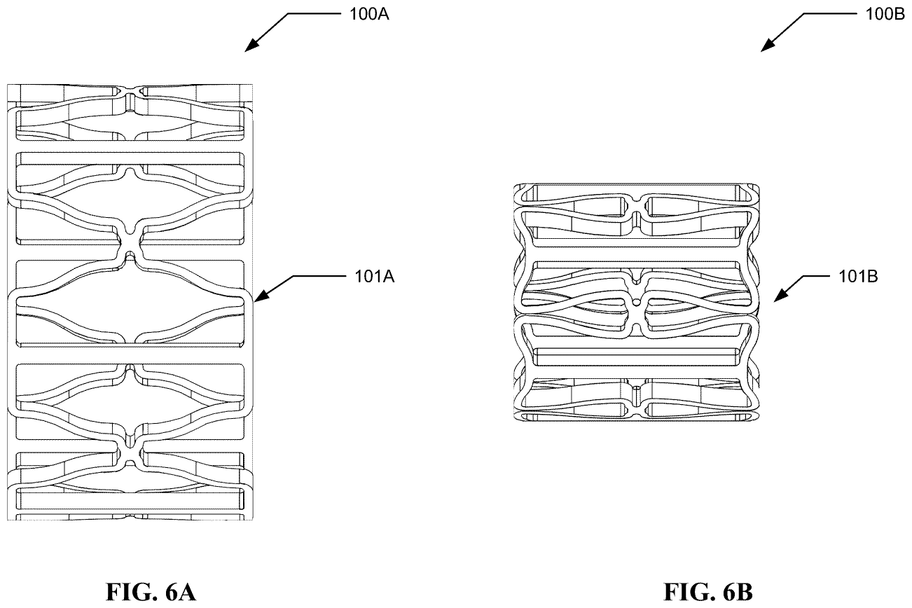

FIG. 6A shows a bottom view of the example stent 100A in an expanded state. FIG. 6B shows a bottom view of the example stent 100B in a collapsed state in accordance with some embodiments of the present disclosure.

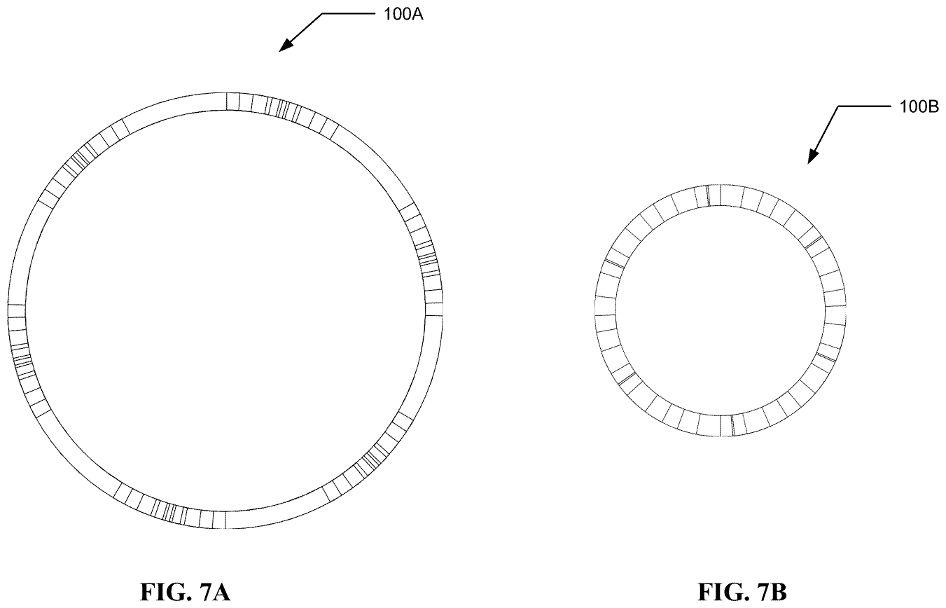

FIG. 7A shows a front view of an example stent 100A in an expanded state. FIG. 7B shows a front view of an example stent 100B in a collapsed state.

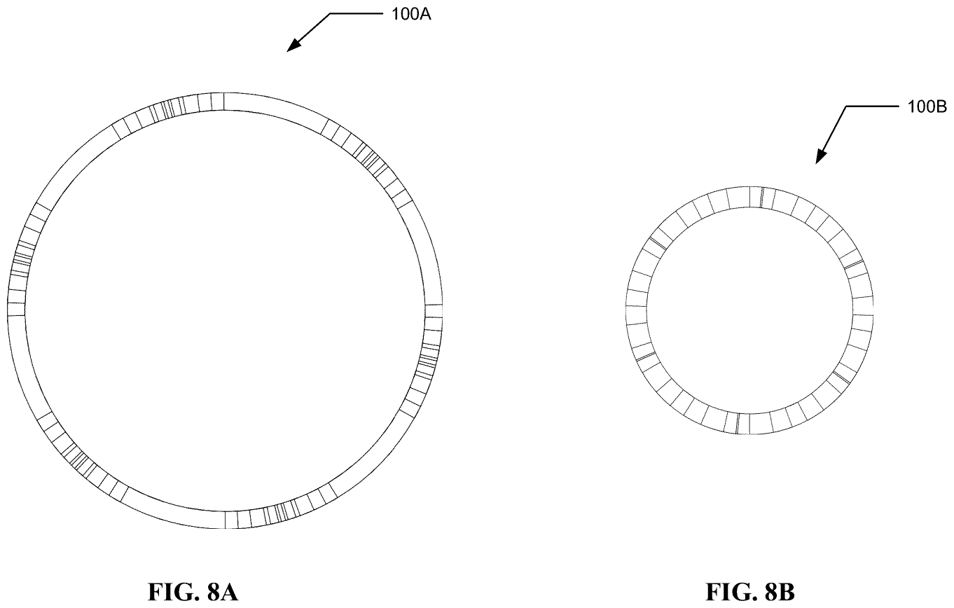

FIG. 8A shows a back view of an example stent 100A in an expanded state. FIG. 8B shows a back view of the example stent 100B in a collapsed state.

FIG. 9 shows an example double-leaf spring 103. In various embodiments, FIG. 9 depicts the double-leaf spring 103 in an expanded state. As described herein, a double-leaf spring may include a first curved arm 105, second curved arm 106, first vertical arm 107, second vertical arm 108, and longitudinal member 109. In some embodiments, a respective curved arm includes a first end 901 and a second end 903 opposite the first end 901. For example, a respective curved arm extends from a first end 901 thereof to a second end thereof. In various embodiments, respective first ends 901 of the first curved arm 105 and second curved arm 106 are connected, said connection being referenced as connection 104 herein and in the accompanying drawings. In some embodiments, a respective vertical arm includes a first end 905 and a second end 907 opposite the first end 905. For example, a respective vertical arm extends from a first end 905 thereof to a second end 907 thereof. In various embodiments, a respective curved arm is connected at the second end 903 to a first end 905 of a vertical arm. For example, the first curved arm 105 is connected at the second end 903 to the first end 905 of the first vertical arm 107.

In some embodiments, the longitudinal member 109 includes a first end 909 and a second end 911 opposite the first end 909. For example, the longitudinal member 109 extends from a first end 909 thereof to a second end 911 thereof. In some embodiments, the longitudinal member includes a first side 913 and a second side 915 opposite the first side 913. In some embodiments, a second end 907 of the first vertical arm 107 is connected on the first side 913 to the first end 909 of the longitudinal member 109. In some embodiments, a second end 907 of the second arm 108 is connected on the first side 913 to the second end 911 of the longitudinal member 109. In various embodiments, a first and second vertical arm of an adjacent double-leaf spring (not shown) are connected, respectively, to the first and second ends 909, 911 on the second side 915 of the longitudinal member 109. For example, the first vertical arms 107 of a pair of adjacent double-leaf springs may form a T-shaped junction with the first end of 909 of the longitudinal member 109 and second vertical arms 108 of the pair of adjacent double-leaf springs may form a T-shaped junction with the second end 911 of the longitudinal member 109. In some embodiments, a first curved arm of another adjacent double-leaf spring (not shown) is connected to the connection 104.

FIG. 10 shows a transition sequence 1000 of an example double-leaf spring between expanded and collapsed states. In various embodiments, the transition sequence 1000 includes an expanded state 1001, an intermediate state 1003, and a collapsed state 1005, where the intermediate state occurs between the expanded state 1001 and the collapsed state 1005. In some embodiments, a respective double-leaf spring is configured to the collapsed state 1005 when covered by a sheath and/or within a catheter of an aspiration apparatus. In some embodiments, upon retraction of the sheath and/or exiting the catheter, the double-leaf spring automatically configures to the intermediate state 1003 and, finally, to the expanded state 1001. In some embodiments, the double-leaf spring is mechanically biased toward configuration to the expanded state 1001.

As described herein and shown in the figures, a self-expanding stent of the present disclosure may include repetitive pattern of leaf-spring elements. A conventional self-expanding stent may expand linearly or an element of the stent works as a typical leaf spring. Conversely, a stent element comprising a curved arm 105, vertical arm 107, and longitudinal member 109 may exhibits a unique non-linearity. Because of the symmetry and repetition, the mechanical performances of a stent may be determined and designed by these fundamental spring elements. As a conventional stent experiences compression force by either blood vessel wall, negative pressure by vacuum, or crimping tools, it shows gradual and steady increases in radial force by the bending action of the leaf spring (e.g., as shown in trend 1107 of FIG. 11). Conversely, in the case of a double-leaf spring of the present disclosure, the radial force increases much faster, reaches the peak and decreases (e.g., as shown in trend 1105 of FIG. 11). This non-linearity is realized by the combination of three spring elements (e.g., curved arm, vertical arm, longitudinal member). In various embodiments, displacement of the curved arm pushes the other vertical spring element sideways (e.g., as shown in the intermediate state 1003).

In some contexts, the curved arm embodies a “weaker leaf-spring” and the vertical arm embodies a “stronger leaf-spring.” When transitioning from the expanded state 1001 to the intermediate state 1003, the forces of the curved arm and vertical arm may be additive. For example, the curved arm may deflect toward the longitudinal member and the vertical arm may deflect outward. As the displacement (e.g., deflection) of the curved arm surpasses a particular point (e.g., the intermediate state 1003), the curved arm begins to pull the vertical spring back (e.g., collapsed state 1005). Here, the some of the forces of the two arms are “subtracted.” For example, the curved arm deflects further toward the longitudinal member and the vertical arm deflects inward relative to its orientation at the intermediate state 1003. This initial additive push produces high spring force in the expanded state 1001 and the subsequent subtractive pull achieves comparatively low spring force in the collapsed state 1005 (e.g., as compared to existing approaches). The movements of the curved arm and vertical arm are supported by the longitudinal backbone. Further, the length of the vertical arm provides space for the leaf-spring to move and accommodate sufficient difference in the maximum and minimum diameters of the double-leaf spring stent, which may determine the diameter of a sheath for delivery and the stent diameter after deployment from the sheath.

In some embodiments, in the expanded state 1001 an angle 1006 between a first curved arm 105 and a first vertical arm 107 is orthogonal. For example, in the expanded state 1001, the first curved arm 105 and the first vertical arm are orthogonal with respect to one another. In some embodiments, in the expanded state 1001, an angle 1008 between the first vertical arm 107 and a longitudinal member 109 is orthogonal (e.g., approximately 90 degrees). Similarly, in the expanded state, the second curved arm 106 and the second vertical arm 108 are orthogonal with respect to one another. It will be appreciated that the foregoing and proceeding descriptions of angles between the first curved arm and first vertical arm and between the first vertical arm and longitudinal member may also describe angular relationships of the second curved arm and second vertical arm and of the second vertical arm and the longitudinal member. For example, while not shown in FIG. 10, an angle between a second curved arm and second vertical arm may be orthogonal when the double-leaf spring is configured to the expanded state 1001.

In some embodiments, an external applied force (e.g., a sheath, catheter, and/or the like) causes the double-leaf spring to transition from the expanded state 1001 to the intermediate state 1003. For example, a double-leaf spring stent may be inserted within a sheath that constricts against the double-leaf spring, thereby applying a force to the respective double-leaf springs of the stent to undergo a transition from the expanded state 1001 to the intermediate state 1003 and, further, to the collapsed state 1005. Additionally, or alternatively, in some embodiments, a temperature of a double-leaf spring may be reduced such that one or more shape memory alloys (SMAs) comprising the spring become deformable, thereby facilitating collapse of the spring from the expanded state. For example, one or more alloys from which a stent is fabricated may be designed such that the stent is shrunk before the introduction of the stent into the body, where body temperature following insertion causes expansion of the stent.

In some embodiments, in the intermediate state 1003, the curved arm 105′ is deflected toward the longitudinal member 109′. The deflection of the curved arm 105′ may increase internal stresses within the first curved arm 105′ and at the connection to the first vertical arm 107′ and second curved arm (not shown) such that the first curved arm 105′ exerts a spring force (e.g., embodied as a radial force when a plurality of double-leaf springs are fabricated in an annular arrangement). In some embodiments, in the intermediate state 1003, the first vertical arm 107′ deflects outward under the external force (and/or a transitive force applied by the first curved arm 105′). In some embodiments, the outward deflection of the first vertical arm 107′ at least partially dissipates the internal stresses within the first curved arm 105′ such that the spring force exerted by the spring arrangement is constrained to a peak value.

In various embodiments, in the intermediate state 1003 and the collapsed states 1005, the longitudinal member 109′, 109″ constrains the respective deflections of the first curved arm 105′, 105″ and first vertical arm 107′, 107″ such that the double-leaf spring demonstrates a non-linear radial force profile in the spring force exerted by the spring arrangement of the first curved arm and first vertical arm. In some embodiments, in the collapsed state 1005, the first curved arm 105″ is deflected further toward the longitudinal member 109″. In some embodiments, as the double-leaf spring transitions from the intermediate state 1003 to the collapsed state 1005, the spring force exerted by the first curved arm 105″ falls below the peak value associated with the spring arrangement of the intermediate state (e.g., while remaining above value associated with the spring arrangement of the expanded state 1001). In some embodiments, the first vertical arm 107″ deflects inward relative to the orientation shown in the intermediate state 1003. In some embodiments, in the collapsed state 1005, an angle 1006′ between the first curved arm 105″ and the first vertical arm 107″ is acute. In some embodiments, in the collapsed state 1005, an angle 1008′ between the first vertical arm 107″ and the longitudinal member 109″ is obtuse.

FIG. 11 illustrates a chart 1100 of example radial force-diameter relationships of a conventional stent and a double-leaf spring stent of the present disclosure. In some embodiments, the chart 1100 measures radial force 1101 exerted by a stent as a function of stent diameter 1103.

In various embodiments, the trend 1107 is associated with a conventional self-expanding stent, such as self-expanding stent comprised of individual (e.g., non-doubled) leaf springs. As shown by the trend 1107, a conventional self-expanding stent may demonstrate a linear relationship between stent diameter 1103 and exerted radial force 1101 between a collapsed state 1005, intermediate state 1003, and expanded state 1001. For example, a conventional self-expanding stent may demonstrate a linear increase in exerted radial force 1101 as stent diameter 1103 decreases. Further, a conventional self-expanding stent may demonstrate a maximum exerted radial force 1111 when configured to a minimum stent diameter.

As shown by the trend 1105, a double-leaf spring stent of the present disclosure may demonstrate a non-linear relationship between stent diameter 1103 and exerted radial force 1101. For example, a double-leaf spring may demonstrate a non-linear increase in exerted radial force between an expanded state 1001 and an intermediate state 1003. As another example, the double-leaf spring may demonstrate a non-linear decrease in exerted radial force between the intermediate state 1003 and the collapsed state 1005. Further, a double-leaf spring of the present disclosure may demonstrate a maximum exerted radial force 1109 when configured to an intermediate stent diameter (e.g., intermediate state 1003). In comparison to a conventional self-expanding stent, the present double-leaf spring stent demonstrates low radial force when the stent is collapsed and high radial force when the stent is expanded. As the double-leaf spring stent is collapsed from the expanded state, the stent compression initially deforms both “weaker leaf-springs” (e.g., curved arms) and “stronger leaf-springs” (e.g., vertical arms). Subsequently, the further compression relaxes only the “stronger leaf-spring,” resulting in the decreases in radial force.

In some embodiments, the maximum exerted radial force 1109 is referred to herein as a peak radial force, which occurs during transition of a double-leaf spring structure between an expanded state 1001 and a collapsed state 1005. In some embodiments, in the expanded state 1001, the spring structure exerts an expanded radial force 1113. In some embodiments, in the collapsed state 1005, the spring structure exerts a collapsed radial force 1115. In some embodiments, the expanded radial force 1113 is less than the collapsed radial force 1115. In various embodiments, the peak radial force is greater than the expanded radial force 1113 and greater than the collapsed radial force 1115.

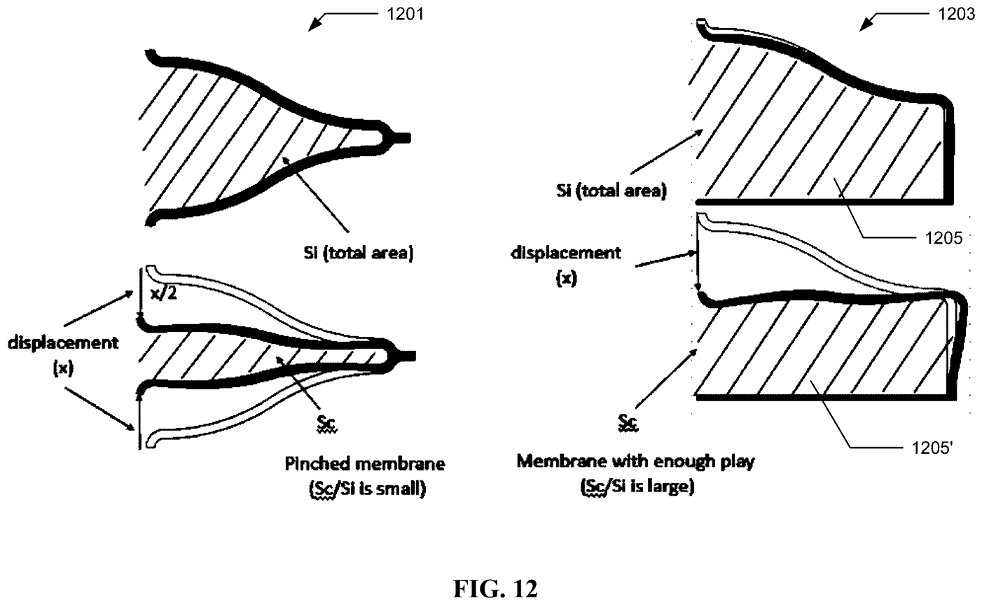

FIG. 12 shows example deformations of a conventional leaf spring stent and a double-leaf spring stent of the present disclosure. In various embodiments, the present double-leaf stent designs present an advantage when a covered stent with this stent structure is used. FIG. 12 demonstrates a comparison of conventional stent deformation 1201 and double-leaf spring stent deformation 1203 with the same displacement to reduce the diameter of the stent. In case of a conventional stent, as the diameter of the stent decreases, the radial force exhibits an inflection point where the radial force increases rapidly. At this inflection point, the membrane becomes “pinched” by the stent struts such that the compression of the membrane exerts excessive radial force making the delivery of the covered stent difficult. To overcome this deficiency, the present double-leaf spring stent reduces this radial force to provide sufficient room for the membrane to keep certain play or being relaxed when the stent is in a sheath catheter. For example, in the case of a non-linear, double-leaf spring stent, the lower part 1205, 1205′ of the structure is undeformed such that it provides ample room for the membrane during the decrease of the stent diameter. Therefore, the inflection point may be well below the diameter when the stent is in the sheath catheter. In doing so, the present double-leaf spring stent provides an added advantage of a relaxed membrane further contributing to the lower radial force during the delivery.

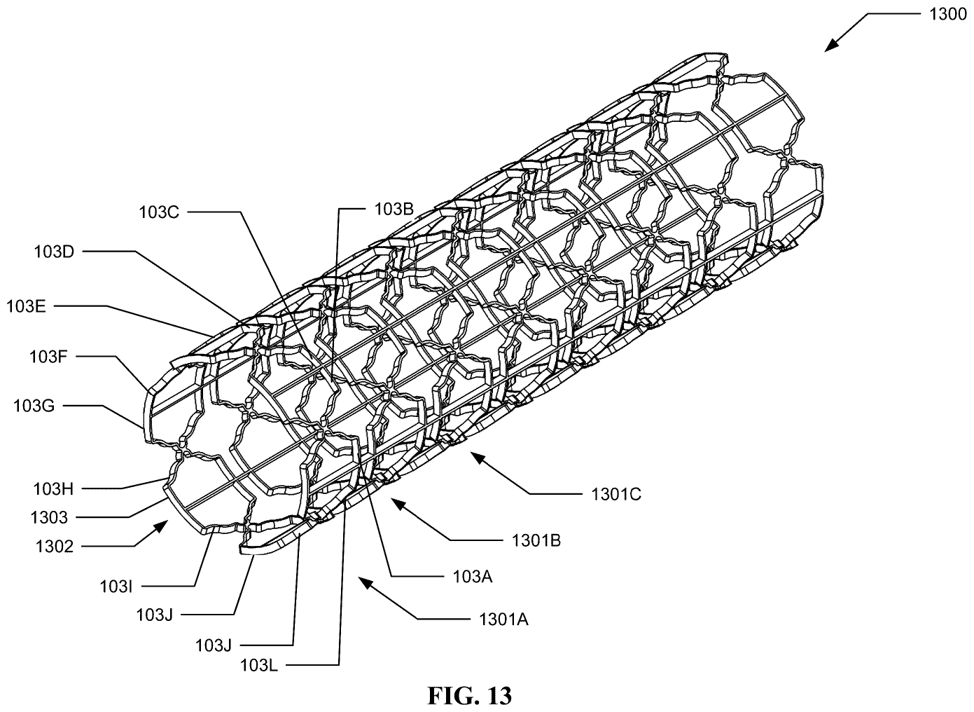

FIG. 13 shows a right perspective view of an example stent 1300 in an expanded state. It will be appreciated that the foregoing description of stent 100A, 100B may apply to the stents 1300, 1900 and similar named elements thereof. In various embodiments, the stent 1300 comprises a plurality of annular sections, where a respective annular section comprises a plurality of double-leaf springs in an annular arrangement. For example, a first annular section 1301A may include a plurality of double-leaf springs 103A-L that are interconnected in an annular shape. In some embodiments, the annular sections of the stent 1300 are interconnected such that the stent is expandable and collapsible via uniform deformation of the annular sections. As one example, the first annular section 1301A may be connected to a first side of a second annular section 1301B. A second side of the second annular section 1301B may be connected to a third annular section 1301C, and further annular sections may be connected to the third annular section and one another in similar fashion.

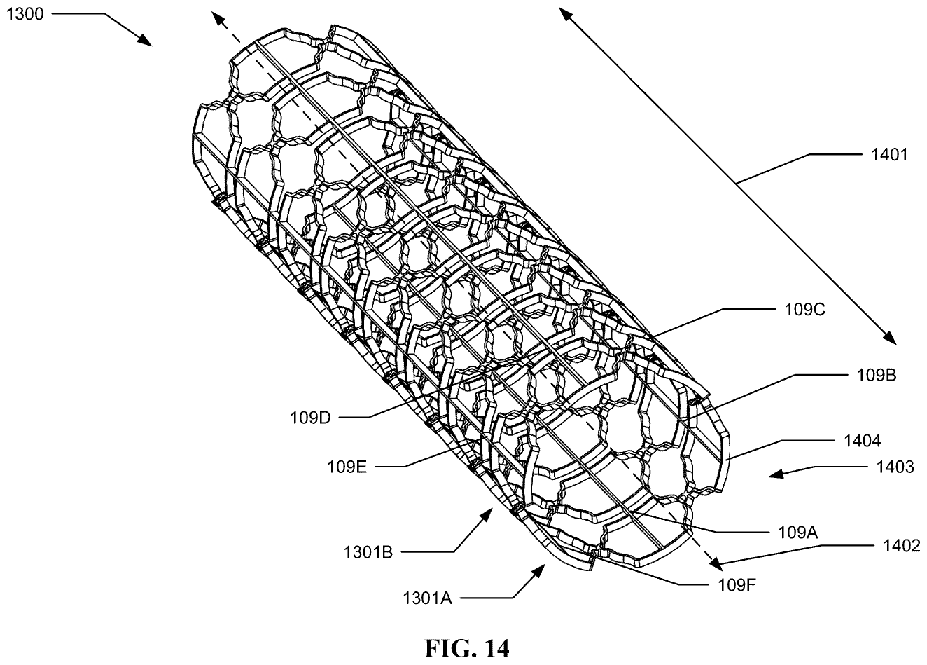

In various embodiments, respective annular sections are connected to one another via the longitudinal members of the double-leaf springs which comprise the sections. For example, as shown in the left perspective view of FIG. 14, the first annular section 1301A may be connected to the second annular section 1301B via longitudinal members 109A-F that extend from a first plurality of double-leaf springs comprising the first annular section 1301A to a second plurality of double-leaf springs comprising the second annular section 1301B. In various embodiments, the longitudinal members 109A-F extend further along a length 1401 of the stent 1300 such that double-leaf springs of subsequent sections comprise respective portions of the longitudinal members 109A-F and such that the subsequent sections are connected via the longitudinal members 109A-F. In various embodiments, as shown in FIG. 14, the stent 1300 is radially symmetrical about a longitudinal axis 1402 extending centrally through the annular sections.

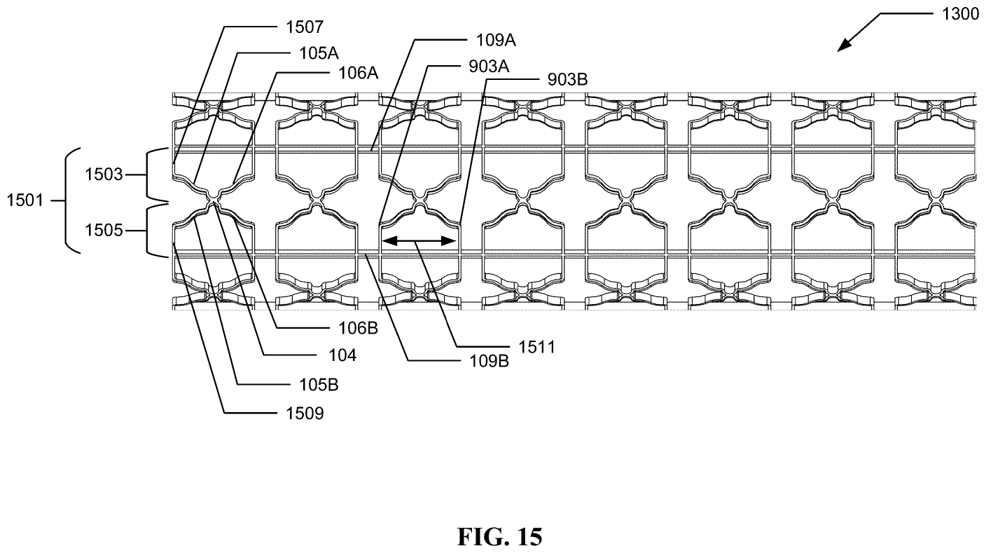

In some embodiments, portions of the connected annular sections define rows of double-leaf springs. For example, the stent 1300 may comprise a plurality of interconnected rows of double-leaf springs in an annular arrangement. As shown in the left-side view of FIG. 15, a respective row 1501 of double-leaf springs may comprise an upper segment 1503 of double-leaf springs and a lower segment 1505 of double-leaf springs. In some embodiments, a respective double-leaf spring 1507 of the upper segment 1503 is connected to an adjacent double-leaf spring 1509 of the lower segment 1505. In some embodiments, respective adjacent double-leaf springs of the upper segment 1503 and the lower segment 1505 are connected via a connection 104 between the curved arms 105A, B, 106A, B of the double-leaf springs.

In some embodiments, the double-leaf springs of a respective segment are connected to a first side of a longitudinal member that defines a length of the row of double-leaf springs. For example, the double-leaf springs of the upper segment 1503 may be connected to a first side of a longitudinal member 109A. A plurality of double-leaf springs comprising a lower segment of an adjacent row may be connected to a second side of the longitudinal member 109A opposite the first side. As another example, the double-leaf springs of the lower segment 1505 may be connected to a first side of a longitudinal member 109B. In various embodiments, a respective connection comprises the longitudinal member and the first and second vertical arms of a respective double-leaf spring. For example, respective second ends 903A, 903B of the curved arms of a double-leaf spring may define a span 1511 of the double-leaf spring. Further, the span 1511 may define a span of the longitudinal member 109B that comprises a portion of the double-leaf spring. The first and second vertical arms of the double-leaf spring may be connected to the longitudinal member 109B at opposing ends of the span 1511.

In some embodiments, as shown in FIG. 13, a first end 1302 of the stent 1300 comprises a first annular arrangement of flat surfaces 1303 spaced apart from one another. In some embodiments, a respective flat surface 1303 of the first annular arrangement is defined by the respective first vertical arm 107 and/or respective second vertical arm 108 of the plurality of double-leaf springs in the first annular section 1301A of the plurality of annular sections. In some embodiments, as shown in FIG. 14, a second end 1403 of the stent 1300 comprises a second annular arrangement of flat surfaces 1404 spaced apart from one another. In various embodiments, a respective flat surface 1404 of the second annular arrangement is defined by the respective first vertical arm 107 and/or respective second vertical arm 108 of the plurality of double-leaf springs in an annular section at the second end 1403. In some embodiments, the flat surfaces 1303, 1404 reduce a risk of piercing tissue, membranes, and/or the like, as the stent 1300 is navigated to and from a target site. For example, existing stents may include wedge- or prong-like arrangements their respective first and/or second ends. The wedge- or prong-like structures may increase act as sharp points and/or pressure concentrations, increasing a likelihood of the stent piercing a delivery membrane (e.g., sheath), a blood vessel wall, and/or the like. In some embodiments, the flat surfaces 1303, 1404 embodying the first end 1302 and second end 1403 of the stent 1300 reduce a likelihood of piercing due to the planar structure and distribution of pressure along the surfaces.

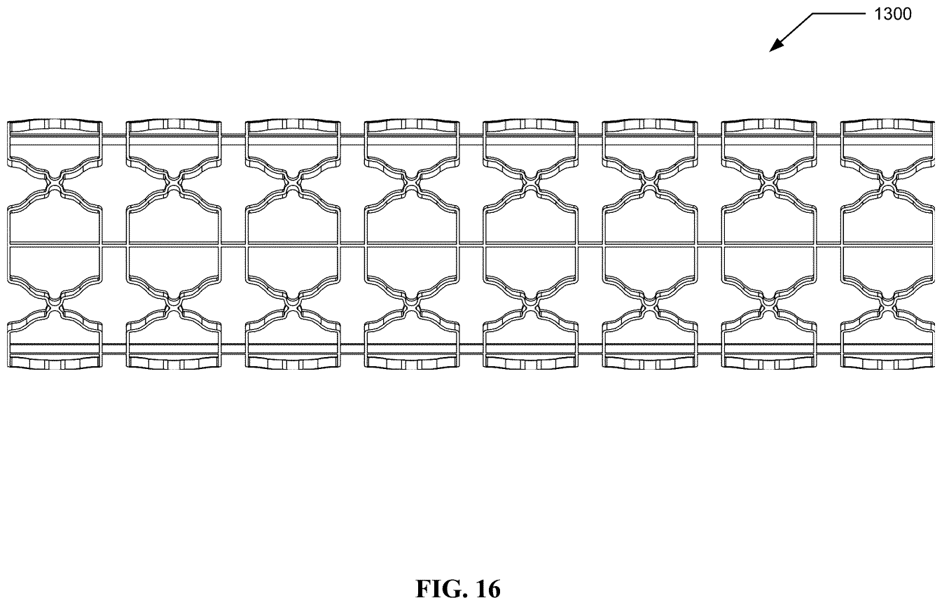

FIG. 16 shows a right-side view of an example stent 1300 in an expanded state.

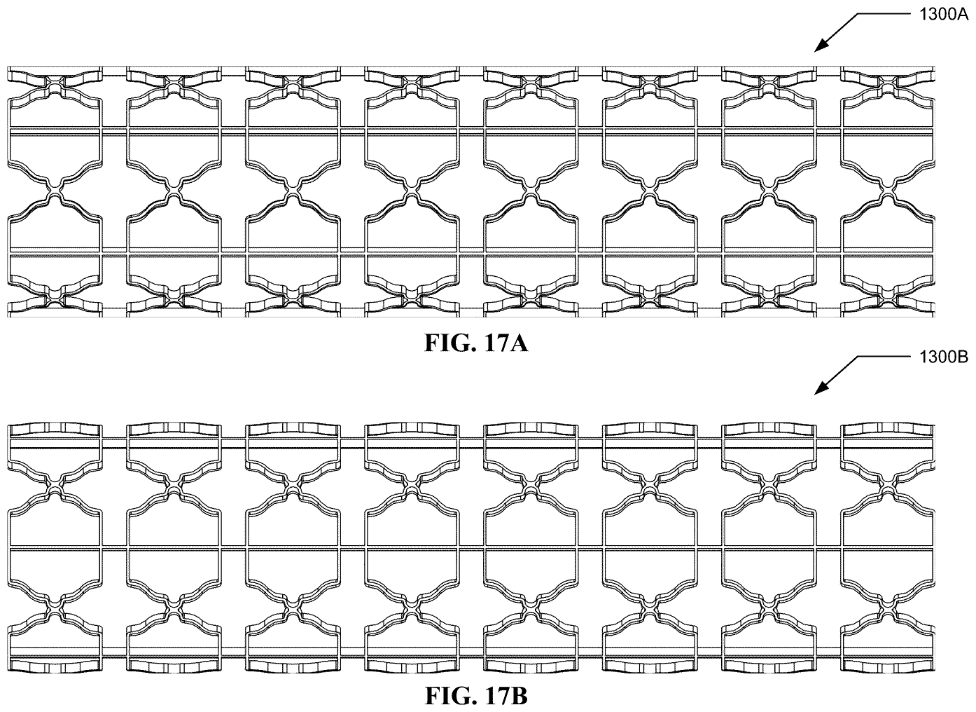

FIGS. 17A-B show, respectively, a top view and a bottom view of an example stent 1300A, 1300B in an expanded state.

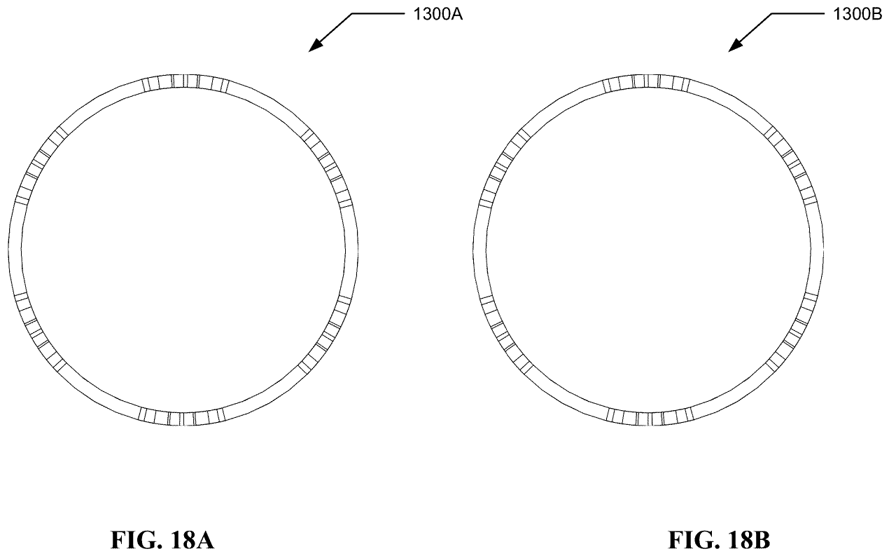

FIGS. 18A-B show, respectively a front view and a back view of an example stent 1300A, 1300B in an expanded state.

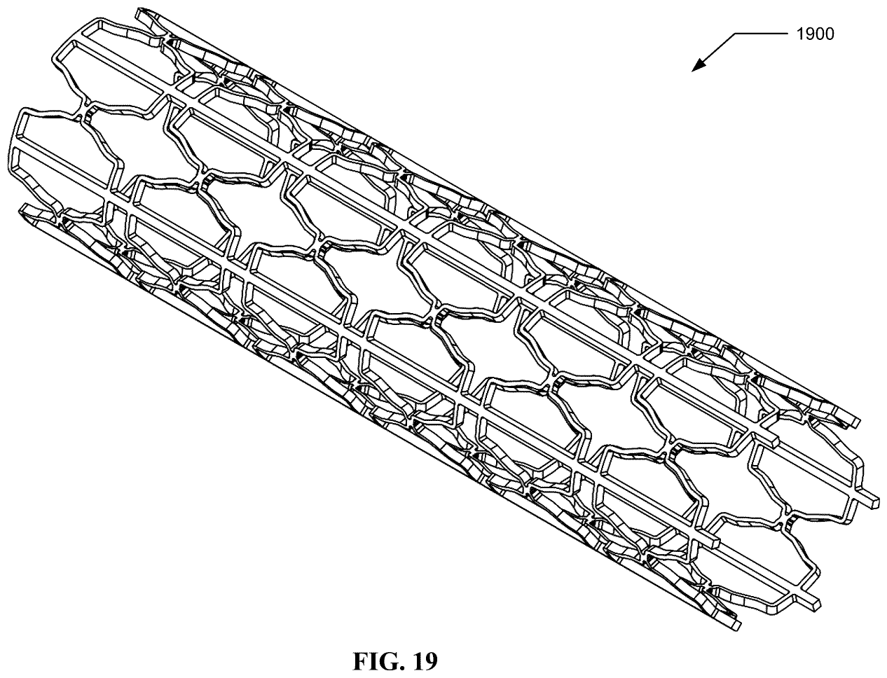

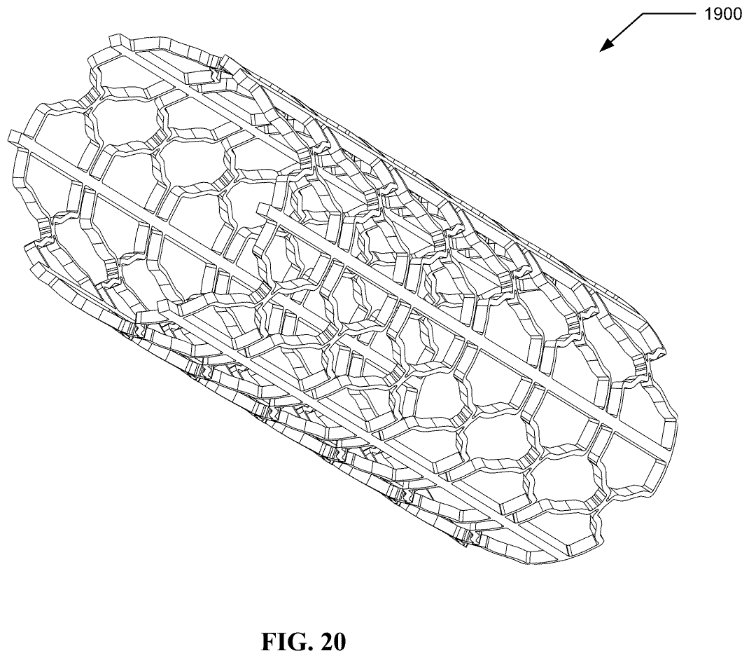

FIG. 19 shows a right perspective view of an example stent 1900 in an expanded state. FIG. 20 shows a left perspective view of an example stent 1900 in an expanded state.

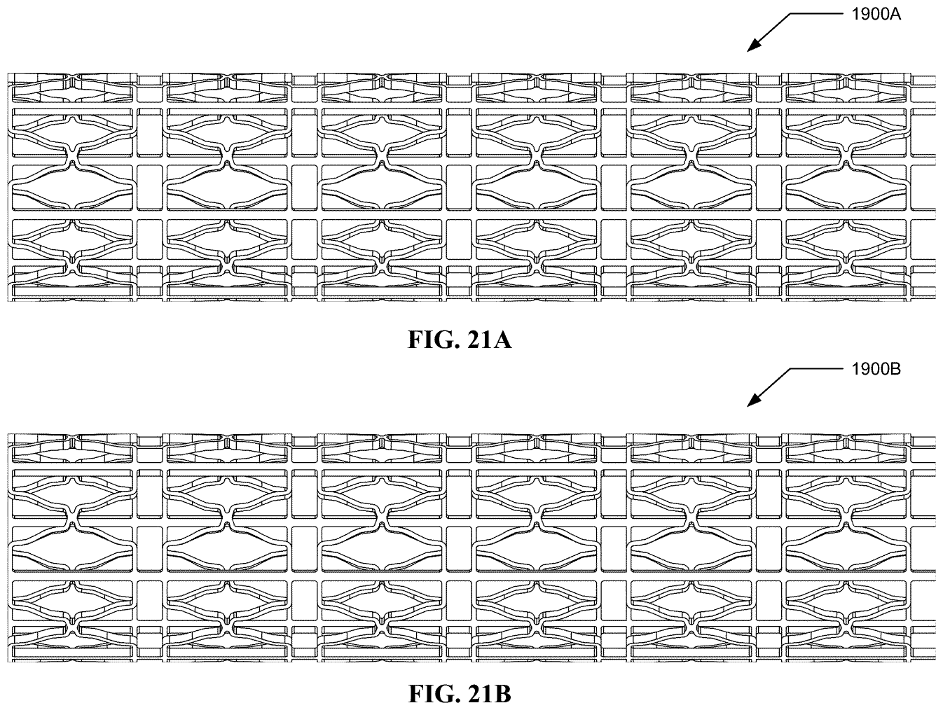

FIGS. 21A-B show, respectively, a left-side and a right-side view of an example stent 1900A, 1900B in an expanded state.

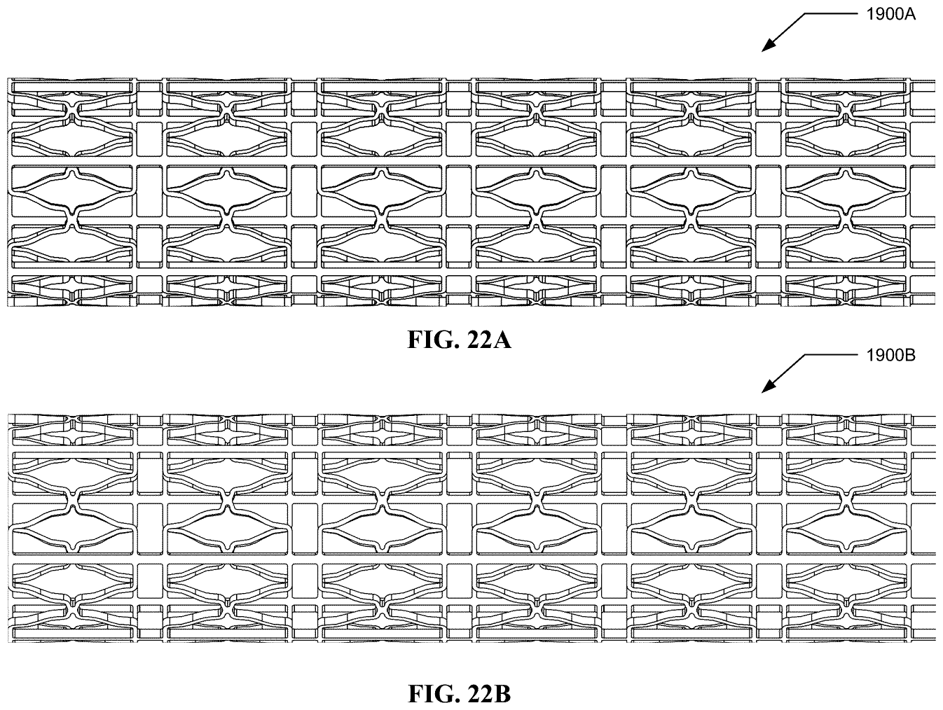

FIGS. 22A-B show, respectively, a top view and a bottom view of an example stent 1900A, 1900B in an expanded state.

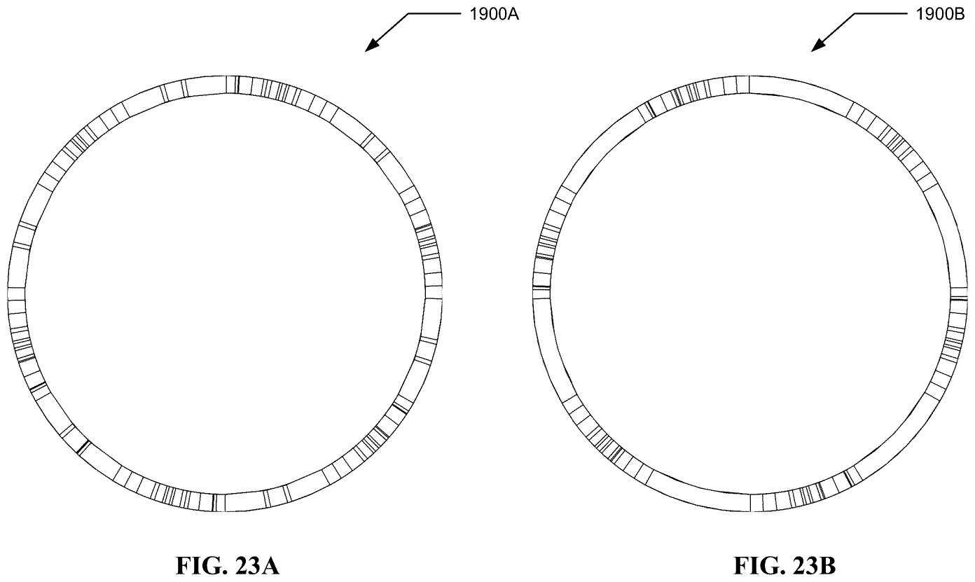

FIGS. 23A-B show, respectively a front view and a back view of an example stent 1900A, 1900B in an expanded state.

### Example Method of Use

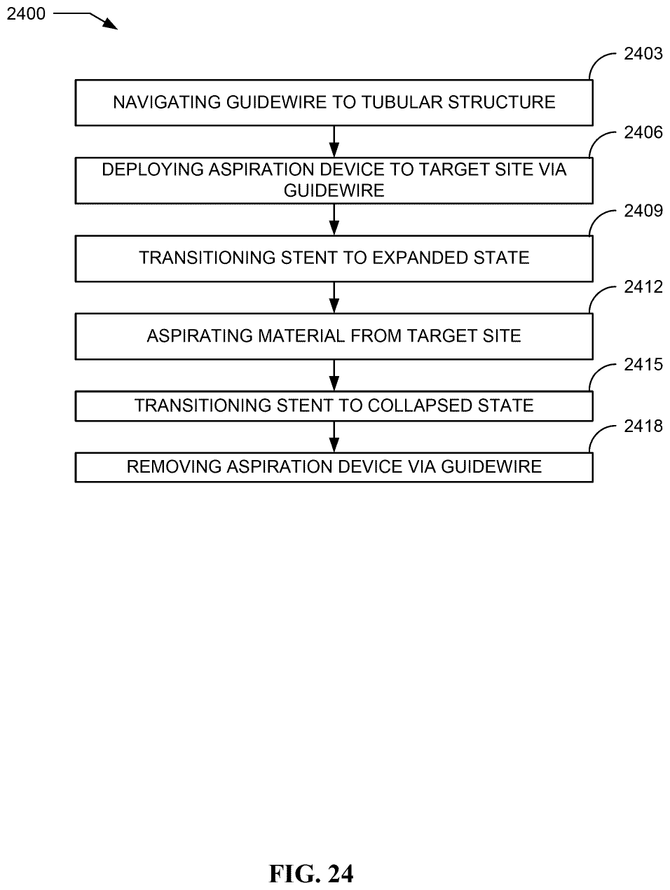

Having described example expandable stents in accordance with the disclosure, example processes of the disclosure will now be discussed. It will be appreciated that the flowchart depicts an example process that is performable using one or more of the expandable stents described herein. For example, an aspiration process 2400 depicted in the flowchart of FIG. 24 and described herein may be performed using one or more stents 100A, B, 1300, 1800, as shown in FIGS. 1A-B, 13, and 19, respectively, and described herein. In some embodiments, one or more processes are performed using a kit comprising one or more stents. For example, the aspiration process 2400 may be performed using a kit comprising one or more stents 100A, B, 1300, 1900, and/or the like. In some embodiments, the kit further includes one or more sheathes configured to cover a respective stent to maintain the stent in a collapsed state, where extraction of the sheath results in transition of the stent from the collapsed state to an expanded state.

In some embodiments, the kit comprises at least a first stent and a second stent. The first stent may comprise a first diameter in an expanded state, and the second stent may comprise a second diameter in an expanded state. The second diameter may be less than, equal to, or greater than the first diameter. Additionally, or alternatively, in some embodiments, one or more double-leaf springs of the first stent comprise a first thickness and one or more double-leaf springs of the second stent comprise a second thickness. In various embodiments, the second thickness is less than, greater than, or equal to the first thickness.

In some embodiments, the kit comprises one or more aspiration devices comprising a respective stent and sheath. In some embodiments, the kit includes one or more sterile covers configured to receive one or more kit components and provide a barrier between an internal cavity of the cover and an external environment.

The depicted blocks indicate operations of each process. Such operations may be performed in any of a number of ways, including, without limitation, in the order and manner as depicted and described herein. In some embodiments, one or more blocks of any of the processes described herein occur in-between one or more blocks of another process, before one or more blocks of another process, in parallel with one or more blocks of another process, and/or as a sub-process of a second process. Additionally, or alternatively, any of the processes in various embodiments include some or all operational steps described and/or depicted, including one or more optional blocks in some embodiments. With regard to the flowcharts illustrated herein, one or more of the depicted block(s) in some embodiments is/are optional in some, or all, embodiments of the disclosure. It should be appreciated that one or more of the operations of each flowchart may be combinable, replaceable, and/or otherwise altered as described herein.

FIG. 24 illustrates a flowchart depicting operations of an example process 2400 for aspirating material from a target site within a tubular structure. For example, the process 2400 may be performed to aspirate a thrombus from a target site within a vein, artery, and/or the like (e.g., thrombectomy). As another example, the process 2400 may be performed to aspirate plaque from a target site (e.g., atherectomy).

In some embodiments, at block 2403, the process 2400 includes navigating a guidewire to a tubular structure of a subject. For example, a guidewire may be inserted into an artery of a subject via an incision and navigated through the artery to a particular section thereof or to a secondary blood vessel (e.g., other artery, vein, and/or the like).

In some embodiments, at block 2406, the process 2400 includes deploying an aspiration device to a target site within the tubular structure via the guidewire. In some embodiments, the aspiration device comprises an expandable catheter and a self-expanding double-leaf spring stent within the catheter, where the self-expanding double-leaf spring stent is covered by a retractable sheath. In some embodiments, the double-leaf spring stent is configured to and maintained in a collapsed state via the sheath cover. In some embodiments, the aspiration device is deployed to a target site via pushing the expandable catheter along the guidewire to a particular portion within the tubular structure that embodies the target site. In some embodiments, the aspiration device includes an aspiration catheter operatively connected to a pump system and/or the like that creates negative pressurization within the aspiration device such that material may be aspirated into the aspiration catheter.

As described herein, in the collapsed state, the double-leaf spring stent may demonstrate a reduced radial force as compared to typical stents for performing aspiration thrombectomies. In various embodiments, the reduced radial force reduces friction of the aspiration device against tubular structures of the subject. The reduced friction may improve the safety and deliverability of the aspiration device as compared to existing approaches. For example, the reduced friction may increase the maneuverability of the aspiration device through blood vessels. As another example, the reduced friction may reduce a pushing force needed to advance the aspiration device, which may reduce a likelihood of piercing or otherwise damaging tissues of the subject.

In some embodiments, at block 2409, the process 2400 includes transitioning a respective double-leaf spring stent of the aspiration device from a constrained state to an expanded state. In some embodiments, the body temperature of the subject results in a transfer of thermal energy to the one or more shape memory materials comprising the double-leaf spring stent. In some embodiments, the thermal transfer raises the temperature of the shape memory material such that the spring structures of the stent undergo a shape memory transition from a constrained toward an expanded state. Additionally, or alternatively, in some embodiments, transitioning the double-leaf spring stent from the constrained state to the expanded state includes retracting a sheath covering the stent. For example, a technician or surgeon performing the process 2400 may trigger a pull mechanism and/or the like that retracts the sheath from the stent, thereby enabling expansion of the stent from the collapsed state.

In various embodiments, the transition of the double-leaf spring stent from the collapsed state to the expanded state significantly increases a bore size of the aspiration device. The increased bore size may be configured for receiving a thrombus, plaque, foreign material, and/or the like, at the target site. In some embodiments, the expansion of the double-leaf spring stent increases the bore size of the aspiration device to approximately the diameter of the tubular structure within which the aspiration device is deployed.

In some embodiments, at block 2412, the process 2400 includes aspirating material from the target site via the aspiration device. For example, a pump system and/or the like may be activated to generate negative pressure within an aspiration catheter of the aspiration device. The negative pressurization of the aspiration catheter may generate a vacuum force at the bore of the aspiration device. The vacuum force may draw thrombi, plaque, foreign material, and/or the like through the expanded double-leaf spring stent into the bore of the aspiration device and, further, into the aspiration device such that the material(s) may be removed from the subject. As described herein, the double-leaf spring stent may demonstrate sufficient radial force in the expanded state such that the stent withstands the negative pressure of the aspiration catheter.

In some embodiments, at block 2415, the process 2400 includes transitioning the respective double-leaf spring stent of the aspiration device from the expanded state to a constrained state. In some embodiments, transitioning the double-leaf spring stent from the expanded state to the constrained state comprises retracting the stent into a sheath such that the sheath covers and compresses the stent into the collapsed state. Alternatively, or additionally, in some embodiments, the sheath is advanced over the stent such that the sheath covers and causes compression of the stent into the collapsed state.

In some embodiments, at block 2418, the process 2400 includes removing the aspiration device from the tubular structure and subject via the guidewire. In some embodiments, removal of the guidewire may follow or occur simultaneous to removal of the aspiration device.

### CONCLUSION

While some embodiments described herein relate to self-expanding stents for aspiration-based thrombectomy, one of ordinary skill in the art will appreciate that the teachings herein may also apply to a wide range of medical procedures and apparatuses. The embodiments described herein may also be scalable to accommodate at least the aforementioned applications. Various components of embodiments described herein can be added, removed, reorganized, modified, duplicated, or the like as one skilled in the art would find convenient and/or necessary to implement a particular application in conjunction with the teachings of the present disclosure. In some embodiments, specialized features, characteristics, materials, components, and/or equipment may be applied in conjunction with the teachings of the present disclosure as one skilled in the art would find convenient and/or necessary to implement a particular application.

Moreover, many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of any appended claims. Moreover, although the foregoing descriptions and the associated drawings describe example embodiments in the context of certain example combinations of elements and/or functions, it should be appreciated that different combinations of elements and/or functions can be provided by alternative embodiments without departing from the scope of any appended claims. In this regard, for example, different combinations of elements and/or functions than those explicitly described above are also contemplated as can be set forth in some of any appended claims. Although specific terms are employed herein, they are used in a generic and descriptive sense only and not for purposes of limitation.

That which is claimed is:

1. A stent, comprising a spring structure, the spring structure configured to:
exert a collapsed radial force in a collapsed state;
exert an expanded radial force in an expanded state, the collapsed radial force being greater than the expanded radial force; and
exert a peak radial force during a transition of the spring structure between the collapsed state and the expanded state, wherein the peak radial force is greater than the collapsed radial force and greater than the expanded radial force.

2. The stent of claim 1, wherein:
the spring structure comprise a plurality of double-leaf springs.

3. The stent of claim 2, wherein:
a respective double-leaf spring comprises:
a first curved arm;
a second curved arm;
a connection between respective first ends of the first curved arm and the second curved arm;
a first vertical arm connected to a second end of the first curved arm;
a second vertical arm connected to a second end of the second curved arm; and
a longitudinal member connected to opposing ends of the first vertical arm and the second vertical arm.

4. The stent of claim 3, wherein:
on a first side, the respective double-leaf spring is connected to an adjacent double-leaf spring via the longitudinal member.

5. The stent of claim 4, wherein:
the longitudinal member of the respective double-leaf spring is connected, at the opposing ends, to a first vertical arm and a second vertical arm of the adjacent double-leaf spring.

6. The stent of claim 4, wherein:
on a second side opposite the first side, the respective double-leaf spring is connected to a second adjacent double-leaf spring via the connection between the respective first ends of the first curved arm and the second curved arm.

7. The stent of claim 3, wherein:
the first curved arm, second curved arm, first vertical arm, second vertical arm, and longitudinal member are integrally formed.

8. The stent of claim 3, wherein:
in the expanded state, the first vertical arm and the second vertical arm are orthogonal to the longitudinal member.

9. The stent of claim 2, wherein:
the plurality of double-leaf springs are integrally formed.

10. The stent of claim 2, wherein:
the spring structure comprises nitinol.

11. A stent, comprising:
a plurality of rows of double-leaf springs in an annular arrangement; and
a respective row of double-leaf springs comprising an upper segment of double-leaf springs and a lower segment of double-leaf springs, wherein:
a respective double-leaf spring of the upper segment is connected to a corresponding double-leaf spring of the lower segment;
the double-leaf springs of a respective segment are connected to a first side of a longitudinal member that defines a length of the respective row; and
the longitudinal member is connected to respective double-leaf springs of a respective upper or lower segment of another row of double-leaf springs.

12. The stent of claim 11, wherein:
a respective double-leaf spring comprises:
a first curved arm;
a second curved arm;
a connection between respective first ends of the first curved arm and the second curved arm, respective second ends of the first curved arm and the second curved arm defining a span of the double-leaf spring;
a first vertical arm connected to the second end of the first curved arm;
a second vertical arm connected to the second end of the second curved arm; and
the respective longitudinal member, wherein the first vertical arm and the second vertical arm are connected to the longitudinal member at opposing ends of the span.

13. The stent of claim 12, wherein:
the annular arrangement is configured to transition the stent between a collapsed state and an expanded state;
in the collapsed state, an angle between a respective vertical arm and the longitudinal member is obtuse; and
in the expanded state, the angle between the respective vertical arm and the longitudinal member is orthogonal.

14. A stent, comprising:
a plurality of annular sections, a respective annular section comprising a plurality of double-leaf springs in an annular arrangement;
a respective double-leaf spring in a first annular section comprising:
a first curved arm;
a second curved arm;
a connection between respective first ends of the first curved arm and the second curved arm, respective second ends of the first curved arm and the second curved arm defining a span of the double-leaf spring;
a first vertical arm connected to the second end of the first curved arm;
a second vertical arm connected to the second end of the second curved arm; and
a longitudinal member, wherein the first vertical arm and the second vertical arm are connected to the longitudinal member at opposing ends of the span; and
the respective longitudinal members of the double-leaf springs in the first annular section extending along a remaining subset of the plurality of the annular sections such that corresponding double-leaf springs in the remaining subset are partially comprised of the longitudinal members.

15. The stent of claim 14, wherein:
a first end of the stent comprises a first annular arrangement of flat surfaces spaced apart from one another; and
a respective flat surface of the first annular arrangement is defined by the respective first vertical arm of the plurality of double-leaf springs in a first annular section of the plurality of annular sections.

16. The stent of claim 15, wherein:
a second end of the stent comprises a second annular arrangement of flat surfaces spaced apart from one another; and
a respective flat surface of the second annular arrangement is defined by the respective second vertical arm of the plurality of double-leaf springs in a second annular section of the plurality of annular sections.

17. The stent of claim 14, wherein:
the stent is radially symmetrical about a longitudinal axis extending centrally through the plurality of annular sections.

18. The stent of claim 14, wherein:
the stent comprises a non-linear radial force profile.

19. The stent of claim 14, wherein:
the stent comprises at least one of cupro nickel aluminum alloy, silico manganese alloy, or cupro zinc aluminum alloy.

20. The stent of claim 14, wherein:
in a respective double-leaf spring:
the first curved arm defines a first leaf spring;
the second curved arm defines a second leaf spring;
the first vertical arm defines a first vertical spring; and
the second vertical arm defines a second vertical spring.
